(19) United States
(12) Patent Application Publication
(10) Pub. No.: US 2011/0118849 A1
(43) Pub. Date: May 19, 2011

(54) Modular Hip Joint Prosthesis and Assembling Method Thereof

(75) Inventors: Tain-Hsiung CHEN, Taipei (TW); Cheng-Kung CHENG, Taipei (TW); Hung-Wen WEI, Taipei (TW); Yu-Shu LAI, Taipei (TW)

(73) Assignees: NATIONAL YANG MING UNIVERSITY, Taipei (TW); TAIPEI VETERANS GENERAL HOSPITAL, Taipei (TW)

(21) Appl. No.: 12/624,251

(22) Filed: Nov. 23, 2009

(30) Foreign Application Priority Data
Nov. 17, 2009 (TW) 098139003

(51) Int. Cl. A61F 2/32; B23P 11/00

(52) U.S. Cl. 623/22.42; 29/428

(57) ABSTRACT

The invention relates to modular hip joint prosthesis and an assembling method thereof. The modular hip joint prosthesis includes a stem component, a proximal body module and lock component. The proximal body module assembles with the stem component and includes a first curved surface component and a second curved surface, wherein the radius of curvature of the first curved surface component is different from the radius of curvature of the second curved surface component. The lock component is used to encase the first curved surface component and the second curved surface for locking up both components.

### BACKGROUND OF THE INVENTION

1. Field of the Invention

The present invention relates to a modular hip joint prosthesis, and more particularly, to a modular hip joint prosthesis and the assembling method thereof.

2. Prior art

when hip joint diseases result in femoral head cartilage erosion or subluxation and hip joint is not capable of normal exercise, total hip joint replacement is usually conducted to rebuild the normal function of the hip joint.

Due to the fact that the sizes of the human intramedullary canals differ depending on races, ages, genders or diseases. Traditional standard hip joints are designed with various appearances and sizes to meet the needs of different patients. Nowadays, modular hip joints have become the solution of meeting the needs of both product inventory, and different sizes and appearances of intramedullary canals.

Nowadays the hip joint body part design (the component between the femoral head and the distal femoral) of the modular hip joint is symmetrical on the appearance. However, the appearance of the proximal intramedullary canal is asymmetrical. To implant the modular hip joint, surgical instrument is used to cut the bones of intramedullary canal to suit the appearance of the hip joint.

Among the hip joint diseases, the patients of Developmental Dysplasia of Hip (DDH) suffer from serious difference of the intramedullary canals. In clinical research, the disadvantage of DDH patients adopting the modular hip joint is that a great deal of femoral bones must be cut off. As a result, hip joint body part does not well match the appearance of the intramedullary canal and the hip joint may depart easily after the surgery.

### SUMMARY OF THE INVENTION

The object of the present invention is to provide a modular hip joint prosthesis and the assembling method thereof in order to improve the disadvantages of the existing techniques.

According to one aspect of the present invention, a modular hip joint prosthesis is provided. The modular hip joint prosthesis comprises a stem component, a proximal body module, and a lock component. The proximal body module connects to the stem component, and the proximal body module further comprises the first curved surface component and the second curved surface component, wherein the curvature radius of the first curved surface component is different from the curvature radius of the second curved surface component. The lock component encases the first curved surface component and the second curved surface component for locking up the first curved surface component and the second curved surface component.

Based on another aspect of the present invention, an assembling method of the modular hip joint prosthesis is provided, comprising: connecting the first curved surface component and the second curved surface component to form a through hole, wherein the curvature radius of the first curved surface component is different than that of the second curved surface component; encasing a stem component into the through hole, wherein the stem component has a first connecting part, and the first curved surface component contains a plurality of second connecting parts; connecting the first connecting part to a different second connecting part depending on a preset anterior tilt angle; and encasing a lock component for locking up the first curved surface component and the second curved surface component.

The advantage of the present invention is that by assembling two or more curved surfaced components, a single stem body component is formed which leads to a more perfect match with the appearance of the intramedullary canal. In addition, a variety of the hip joint appearances can also be created by using the limited modular curved surface components. As a result, the clinical problem of low match between the hip joint body part and the intramedullary canal can be solved, and more various body part appearances can be created by using limited modular components.

The foregoing aspects and many of the attendant advantages of this invention will become more readily appreciated as the same becomes better understood by reference to the following detailed description, when taken in conjunction with the accompanying drawings.

### BRIEF DESCRIPTION OF THE DRAWINGS

FIG. 1 is the chart of the modular hip joint according to the preferred embodiment of the present invention.

FIG. 2 is the decomposition of the modular hip joint according to the preferred embodiment of the present invention.

FIG. 3 is another decomposition of the modular hip joint according to the preferred embodiment of the present invention.

FIG. 4a is the chart showing the preset anterior tilt angle is zero according to the preferred embodiment of the present invention.

FIG. 4b is the chart showing the preset anterior tilt angle is 30 degree according to the preferred embodiment of the present invention.

FIG. 5 is the flowchart of the assembling method of the modular hip joint prosthesis according to the preferred embodiment of the present invention.

### DETAILED DESCRIPTION OF THE PREFERRED EMBODIMENT

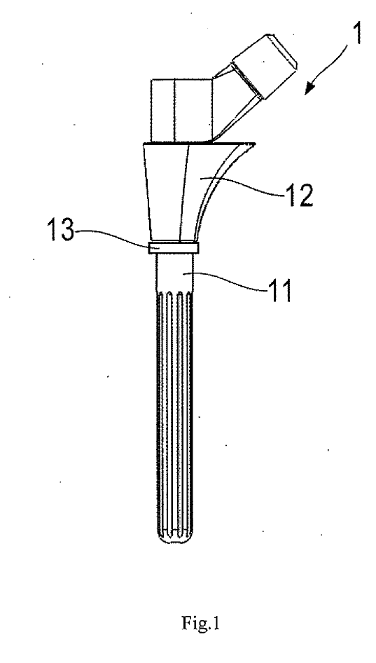

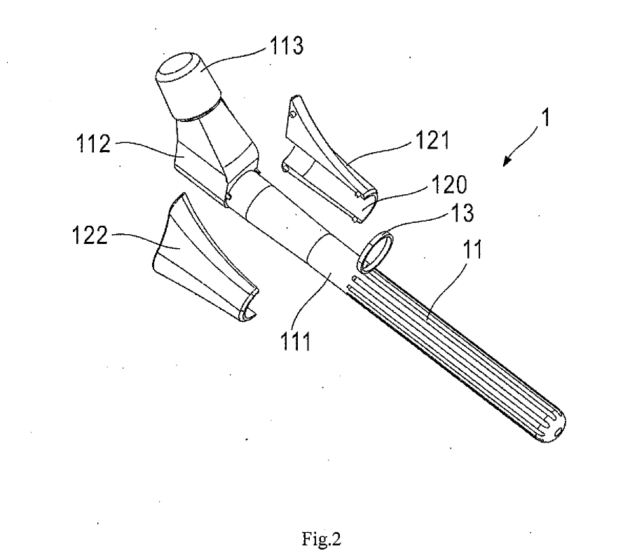

FIG. 1 is the chart of the modular hip joint according to the preferred embodiment of the present invention. FIG. 2 is the decomposition of the modular hip joint according to the preferred embodiment of the present invention. Detailed description of the preferred embodiment of the present invention will conduct with FIG. 1, and FIG. 2.

The modular hip joint 1 provided in this embodiment comprises the stem component 11, the proximal body module 12 and the lock component 13, wherein the stem component 11 further comprises the remote part 111, the turning part 112, and the head part 113. The proximal body module 12 also comprises the first curved surface component 121 and the second curved surface component 122.

In the preferred embodiment, the curvature radius of the first curved surface component 121 is different from the curvature radius of the second curved surface component 122. In other words, in the preferred embodiment, the shapes of the first curved surface component 121 and the second curved surface component 122 are not symmetrical.

In the preferred embodiment, the first curved surface component 121 can has a plurality of clipping parts (not shown in fig.), and the second curved surface component 122 can contain a plurality of connecting holes (not shown in fig.) so that the first curved surface component 121 and the second curved surface component 122 can be conveniently connected without being departed easily. However, the present invention does not limit the connecting method of the first curved surface component 121 and the second curved surface component 122. In addition, when the first curved surface component 121 is connected to the second curved surface component 122, a through hole 120 is formed. The aforementioned stem component 11 can go through the through hole 120 so that the proximal body module 12 and the stem component 11 can be connected.

One end of the aforementioned turning part 112 couples to the remote part 111, and the other end of the turning part 112 couples to the head part 113. The lock component 13 is used to encase the first curved surface component 121 and the second curved surface component 122 for locking up the first curved surface component 121 and the second curved surface component 122 on the stem component 11.

In the preferred embodiment, the material of the lock component 13 can be elastic resin or other materials which can be disposed inside the human body. The lock component 13 can be encased on the ends of the first curved surface component 121 and the second curved surface component 122 to avoid the first curved surface component 121 and the second curved surface component 122 from departing. In other embodiments, the lock component 13 has threads, and there are also threads on the first curved surface component 121 and the second curved surface component 122 so that the lock component 13 can be locked on the first curved surface component 121 and the second curved surface component 122. The present invention does not limit the locking relationship between the lock component 13, and the first curved surface component 121 and the second curved surface component 122.

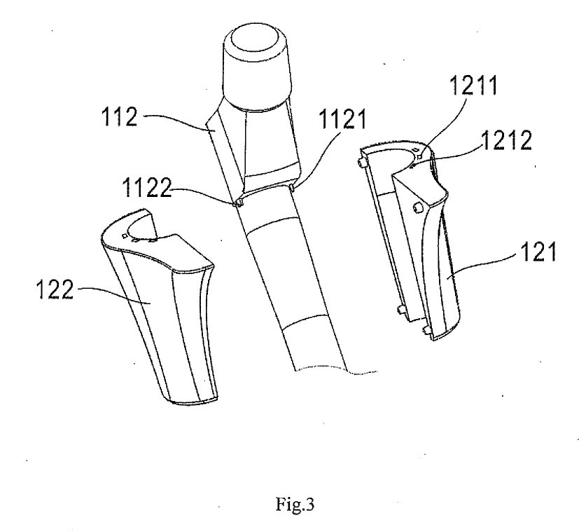

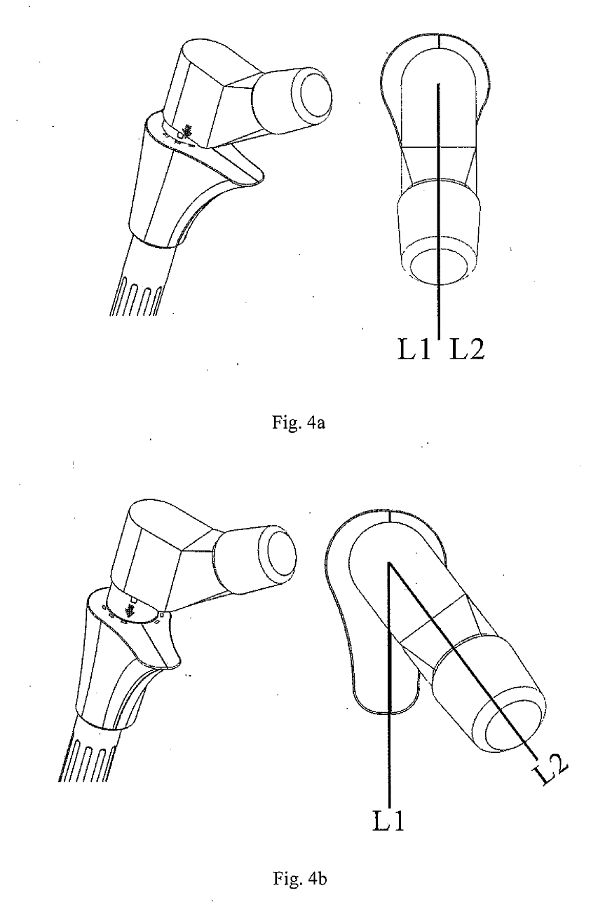

FIG. 3 is another decomposition of the modular hip joint according to the preferred embodiment of the present invention. FIG. 4a is the chart showing the preset anterior tilt angle is zero according to the preferred embodiment of the present invention. FIG. 4b is the chart showing the preset anterior tilt angle is 30 degree according to the preferred embodiment of the present invention.

Referring to FIG. 3, FIG. 4a and FIG. 4b, in the preferred embodiment, the aforementioned turning part 112 has the first convex 1121 and the second convex 1122. The first curved surface component 121 has a plurality of first shrinkage pools 1211 and 1212. The turning part 112 has the first axis L1, and the proximal body module 12 has the second axis L2. There is a preset anterior tilt angle between the first axis L1 and the second axis L2. In the preferred embodiment, the anterior tilt angle can be zero or 30 degree. The present invention does not limit the degree of the preset anterior tilt angle.

In the preferred embodiment, the first convex 1121 can be disposed on one of the first shrinkage pools 1211 or 1212 depending on the preset anterior tilt angle. For example, when the anterior tilt angle is zero, the first convex 1121 can be disposed on the first shrinkage pool 1211; when the preset anterior tilt angle is 30 degree, the first convex 1121 can be disposed on the first shrinkage pool 1212.

In the preferred embodiment, the first convex 1121 can also be referred as the first connecting part. The aforementioned first shrinkage pools 1211 and 1212 can also be referred as a plurality of the second connecting parts. The first connecting part can connect to a different second connecting part depending on the preset anterior tilt angle. The present invention does not limit that the first connecting part shall be a convex. The first connecting part can also be a concave and the second connecting part can be a convex.

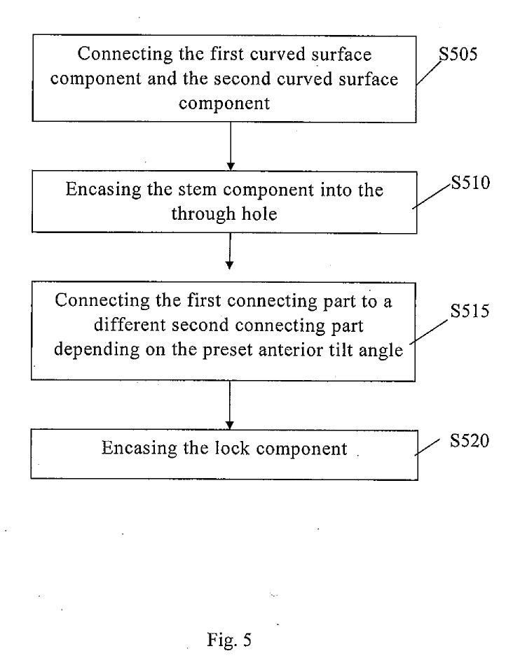

FIG. 5 is the flowchart of the assembling method of the modular hip joint prosthesis according to the preferred embodiment of the present invention. Detailed description of FIG. 5 will conduct with FIG. 2, FIG. 3, FIG. 4a and FIG. 4b.

In step S505, the first curved surface component 121 and the second curved surface component 122 is connected to form the proximal body module 12, and the proximal body module 12 has the through hole 120.

In step S510, encase the stem component 11 into the through hole 120. As aforementioned, the stem component can contain the first connecting part and the first curved surface component can has a plurality of second connecting parts.

In step S515, connect the first connecting part with a different second connecting part depending on the preset anterior tilt angle.

In step S520, encase the lock component 13 on the end of the proximal body module 12 to lock up the first curved surface component 121 and the second curved surface component 122 so that the first curved surface component 121 and the second curved surface component 122 can not be easily departed and the proximal body module 12 is fixed on the stem component 11.

In conclusion, the modular hip joint prosthesis provided in the present invention is to assemble two or more curved surfaced components to form a single stem body component, which leads to a higher match with the intramedullary appearance so that the clinical problem of low match between the hip joint body part and the intramedullary appearance can be solved. In addition, more various body appearances can also be created by using limited modular components.

While the preferred embodiment of the invention has been illustrated and described, it will be appreciated that various changes can be made therein without departing from the spirit and scope of the invention.

What is claimed is:

1. A modular hip joint prosthesis, comprising:
a stem component;
a proximal body module, which connects to the stem component, the proximal body module further comprises a first curved surface component and a second curved surface component; and
a lock component, which encases the first curved surface component and the second curved surface component for locking up the first curved surface component and the second curved surface component;
wherein the curvature radius of the first curved surface component is different from that of the second curved surface component.

2. The modular hip joint prosthesis as claimed in claim 1, wherein the stem component further comprises a turning part with a first convex, the first curved surface component has a plurality of the first shrinkage pools, and the first convex can be disposed on one of the first shrinkage pools depending on the preset anterior tilt angle.

3. The modular hip joint prosthesis as claimed in claim 1, wherein the stem component further comprises a turning part with a first axis, the proximal body module contains a second axis and there is a preset anterior tilt angle between the first and the second axis.

4. The modular hip joint prosthesis as claimed in claim 3, where in the turning part comprises a first connecting part, the first curved surface component contains a plurality of second connecting parts and the first connecting part connects to a different second connecting part depending on the preset anterior tilt angle.

5. The modular hip joint prosthesis as claimed in claim 1, wherein the stem component further comprises a turning part, a head part, and a remote part, one end of the turning part couples to the head part and the other end couples to the remote part.

6. The modular hip joint prosthesis as claimed in claim 1, wherein the first curved surface component and the second curved surface component form a through hole and the stem component goes through the through hole.

7. An assembling method of the modular hip joint prosthesis, comprising:
connecting a first curved surface component and a second curved surface component to form a through hole, wherein the curvature of the first curved surface component is different from the curvature radius of the second curved surface component;
encasing a stem component into the through hole, wherein the stem component comprises a first connecting part, and the first curved surface component comprises a plurality of the second connecting parts;
connecting the first connecting part to a different second connecting part depending on a preset anterior tilt angle; and
encasing a lock component to lock up the first curved surface component and the second curved surface component.

8. The assembling method as claimed in claim 7, wherein the stem component further comprises a turning part with a first axis, and the proximal body module has a second axis, there is a preset anterior tilt angle between the first and the second axis.

9. The assembling method as claimed in claim 7, wherein the stem component further comprises a turning part, a head part, and a remote part, one end of the turning part couples to the head part and the other end couples to the remote part.
